(19) United States
(12) Patent Application Publication
(10) Pub. No.: US 2014/0378782 A1
(43) Pub. Date: Dec. 25, 2014

(54) INTEGRATED RESUSCITATION APPARATUS AND METHOD INCLUDING PERFUSION MONITOR

(71) Applicant: ZOLL MEDICAL CORPORATION, Chelmsford, MA (US)

(72) Inventors: Ulrich Herken, Medford, MA (US); Gary A. Freeman, Newton Center, MA (US)

(21) Appl. No.: 14/313,772

(22) Filed: Jun. 24, 2014

Related U.S. Application Data
(60) Provisional application No. 61/839,233, filed on Jun. 25, 2013.

(51) Int. Cl. A61B 5/026; A61B 5/00; A61B 5/11; A61B 5/1455; A61N 1/39; A61B 5/0205

(52) U.S. Cl. CPC A61B 5/026; A61N 1/3987; A61B 5/0205; A61B 5/11; A61B 5/14551; A61B 5/6823
USPC 600/301; 607/5

(57) ABSTRACT

Disclosed herein are methods and systems of applying treatment to a subject experiencing cardiac distress. In one example, there is provided a resuscitation apparatus. The resuscitation apparatus comprises a displacement monitor, a blood perfusion monitor, and a processor coupled to the displacement monitor and to the blood perfusion monitor. The processor is configured to initiate a measurement of blood perfusion using the blood perfusion monitor responsive to a signal received from the displacement monitor.

### RELATED APPLICATIONS

This application claims priority under 35 U.S.C. §119(e) to U.S. Provisional Application Ser. No. 61/839,233, titled “INTEGRATED RESUSCITATION APPARATUS AND METHOD INCLUDING PERFUSION MONITOR,” filed on Jun. 25, 2013, which is herein incorporated by reference in its entirety.

### BACKGROUND

1. Technical Field

Aspects and embodiments of the present invention relate to methods and systems for the resuscitation of individuals experiencing cardiac distress.

2. Discussion of Related Art

Resuscitation of individuals experiencing cardiac distress can generally include clearing a subject's airway, assisting the subject's breathing, chest compressions, and defibrillation.

The American Heart Association's Basic Life Support for Health Care Providers textbook provides a flow chart at page 4-14 of Chapter 4 that lists the steps of airway clearing, breathing, and circulation (known as A, B, and C), for situations in which there is no defibrillator readily accessible to the rescuer.

Defibrillation (sometimes known as step D) can be performed with the use of an automatic external defibrillator (AED). Most automatic external defibrillators are actually semi-automatic external defibrillators (SAED), which require a clinician to press a start button, after which the defibrillator analyzes the subject's condition and provides a shock to the subject if the electrical rhythm is shockable and waits for user intervention before any subsequent shock. Fully automatic external defibrillators, on the other hand, do not wait for user intervention before applying subsequent shocks. As the term is used below, automatic external defibrillators (AED) include semi-automatic external defibrillators (SAED).

Both AEDs and SAEDs typically provide an oral stand clear warning before the application of each shock. A clinician is expected to stand clear of the subject and may be required to press a button indicating that the clinician is standing clear of the subject. The controls for AEDs are typically located on a resuscitation control box.

AEDs are typically used by trained providers such as physicians, nurses, fire department personnel, and police officers. There might be one or two people at a given facility that has an AED who have been designated for defibrillation resuscitation before an ambulance service arrives. The availability of on-site AEDs along with rescuers trained to operate them is important because if a subject to experiencing cardiac distress experiences a delay of more than about four minutes before receiving a defibrillation shock, the subject's chance of survival can drop dramatically. Many large cities and rural areas have low survival rates for defibrillation because the ambulance response time is slow, although many suburbs have higher survival rates because of the faster ambulance response time due to lack of traffic and availability of hospitals and advanced life support.

Trained lay providers are a new group of AED operators, but they rarely have opportunities to defibrillate. For example, spouses of heart attack victims may become lay providers, but these lay providers can be easily intimidated by an AED during a medical emergency. Consequently, such lay providers can be reluctant to purchase AEDs, or might tend to wait for an ambulance to arrive rather than use an available AED, out of concern that the lay provider might do something wrong.

There are many different kinds of heart rhythms, some of which are considered shockable and some of which are not. For example, a normal heart rhythm is considered non-shockable, and there are also many abnormal non-shockable rhythms. There are also some abnormal non-viable non-shockable rhythms. A subject exhibiting a non-viable non-shockable rhythm cannot remain alive with the non-viable rhythm, yet applying shocks will not help convert the rhythm to a viable rhythm.

As an example of a non-viable non-shockable rhythm, if a subject experiences asystole, the heart will not be beating and application of shocks will be ineffective.

Pacing is recommended for asystole, and there are other things that an advanced life support team can do to assist such subject, such as performing an intervention involving various drugs. The job of the first responder is simply to keep the subject alive, through the use of cardio-pulmonary resuscitation (CPR) and possibly defibrillation, until an advanced life support team arrives. Bradycardias, during which the heart beats too slowly, are non-shockable and also possibly non-viable. If the subject is unconscious during bradycardia, it can be helpful to perform chest compressions until pacing becomes available. Electro-mechanical dissociation (EMD), in which there is electrical activity in the heart but it is not making the heart muscle contract, is non-shockable and non-viable, and would require CPR as a first to response. Idio-ventricular rhythms, in which the normal electrical activity occurs in the ventricles but not the atria, can also be non-shockable and non-viable (usually, abnormal electrical patterns begin in the atria). Idio-ventricular rhythms typically result in slow heart rhythms of 30 or 40 beats per minute, often causing the subject to lose consciousness. The slow heart rhythm occurs because the ventricles ordinarily respond to the activity of the atria, but when the atria stop their electrical activity, a slower, backup rhythm occurs in the ventricles.

The primary examples of shockable rhythms, for which a first responder should perform defibrillation, include ventricular fibrillation, ventricular tachycardia, and ventricular flutter.

After using a defibrillator to apply one or more shocks to a subject who has a shockable electrical rhythm, the subject may nevertheless remain unconscious, exhibiting a shockable or non-shockable rhythm. The rescuer may then resort to chest compressions. As long as the subject remains unconscious, the rescuer can alternate between use of the defibrillator (for analyzing the electrical rhythm and possibly applying a shock) and performing CPR.

CPR generally involves a repeating pattern of five to fifteen chest compressions followed by a pause. CPR is generally ineffective against abnormal rhythms, but it does keep some level of blood flow going to the subject's vital organs until an advanced life support team arrives. It is difficult to perform CPR over an extended period of time. Certain studies have shown that over a course of minutes, rescuers tend to perform chest compressions with less-than-sufficient strength to cause an adequate supply of blood to flow to the brain. CPR prompting devices can assist a rescuer by prompting each chest compression and breath.

### SUMMARY

In accordance with an aspect of the present invention, there is provided a resuscitation device. The resuscitation device comprises a displacement monitor, a blood perfusion monitor, and a processor in communication with the displacement monitor and the blood perfusion monitor. The processor is configured to initiate a measurement of blood perfusion using the blood perfusion monitor responsive to a to signal received from the displacement monitor. In some embodiments, the displacement monitor comprises an accelerometer.

In some embodiments, the resuscitation device is configured to be disposed on a chest of a subject, and the processor is further configured to analyze the signal received from the displacement monitor, determine a point in a CPR compression-relaxation cycle being performed on the subject based upon a result of the analysis, and initiate the measurement of blood perfusion responsive to a determination that the point in the CPR compression-relaxation cycle is at a predefined point. The predetermined point may correspond to the chest of the subject being in a relaxed state.

In some embodiments, the processor is further configured to initiate a second measurement of blood perfusion responsive to a determination that the CPR compression-relaxation cycle is at a second point, the second point corresponding to the chest of the subject being in a compressed state. The processor may be further configured to prompt a CPR administrator to check a pulse of the subject responsive to a result of a comparison between the first measurement of blood perfusion and the second measurement of blood perfusion. The processor may be further configured to prompt a CPR administrator to check a pulse of the subject responsive to a result of the first measurement of blood perfusion.

In some embodiments, the blood perfusion monitor is disposed on a portion of the resuscitation device to which chest compressions are applied during administration of CPR. The blood perfusion monitor may comprise an optical pulse oximetry system. The optical pulse oximetry system may comprise a plurality of optical sensors disposed at different locations on the portion of the resuscitation device to which chest compressions are applied during administration of CPR.

In some embodiments, the blood perfusion monitor comprises a plurality of electrodes configured to electrically contact skin of the subject and the device includes circuitry configured to determine an impedance between at least one pair of the plurality of electrodes.

In some embodiments, the resuscitation device further comprises an electrode pad including a pair of defibrillation electrodes, the displacement monitor, and the to blood perfusion monitor. The displacement monitor, the blood perfusion monitor, and the processor may be integrated in a integral CPR assistance and feedback device.

In accordance with another aspect, there is provided a method of treating a subject using a resuscitation device. The method comprises determining at least one point in a CPR compression-relaxation cycle at which to measure perfusion of the subject, the CPR compression-relaxation cycle including a compression phase and a relaxation phase, monitoring information indicative of displacement of a chest of the subject during performance of the CPR compression-relaxation cycle using the resuscitation device, and measuring the perfusion of the subject using the resuscitation device in response to the monitored information corresponding to the at least one point in the CPR compression-relaxation cycle.

In some embodiments, the at least one point corresponds to the chest of the subject being in a relaxed state. The method may further comprise instructing a CPR administrator to temporarily delay application of another CPR compression-relaxation cycle prior to measuring the perfusion.

In some embodiments, determining the at least one point includes determining a first point corresponding to the chest of the subject being in a relaxed state and determining a second point corresponding to the chest of the subject being in a compressed state.

In some embodiments, the method further comprises prompting a rescuer to check a pulse of the subject responsive to a result of the measurement of perfusion.

In some embodiments, measuring the perfusion of the subject comprises monitoring the perfusion of the subject at a plurality of points in the CPR compression-relaxation cycle. Measuring the perfusion of the subject may further comprise determining a difference in perfusion between at least two of the plurality of points in the CPR compression-relaxation cycle. Measuring the perfusion of the subject may comprise performing a measurement of an impedance across a portion of skin of the chest of the subject.

In some embodiments, the method further comprises delivering a defibrillation shock to the subject.

In some embodiments, measuring the perfusion of the subject comprises monitoring the perfusion of the subject at a plurality of physical locations on the subject. A first physical location of the plurality of physical locations may be proximate a center of a CPR compression region and a second physical location of the plurality of physical locations may be proximate a periphery of the CPR compression region.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A is a drawing of a defibrillation electrode pad positioned over the chest of a subject;

FIG. 1B is a drawing of a defibrillation electrode pad positioned over the chest of a subject in accordance with an embodiment of the present invention;

FIG. 1C is a drawing of a defibrillation electrode pad positioned over the chest of a subject in accordance with an embodiment of the present invention;

FIG. 1D is a drawing of a defibrillation electrode pad positioned over the chest of a subject in accordance with an embodiment of the present invention;

FIG. 2 is a view of the front display panel of a resuscitation control box that houses electronic circuitry and provides audible and visual prompting and which may be used with any of the defibrillation electrode pad of FIGS. 1A-1D;

FIG. 3 is a cross-sectional drawing of the defibrillation electrode pad of FIG. 1A taken along line 3-3;

FIG. 4 is a cross-sectional drawing of the defibrillation pad of FIG. 1A taken along line 4-4;

FIG. 5 is a schematic diagram illustrating the circuit interconnections between the defibrillation electrode pads of FIGS. 1A-1D and the resuscitation control box of to FIG. 2;

FIG. 6 is a block diagram of the electronic circuitry of an alternative embodiment;

FIG. 7 is a drawing of another embodiment of a defibrillation electrode assembly;

FIGS. 8A-8C are diagrammatic views of three embodiments of first and second units;

FIGS. 9A and 9B are drawings of two alternative embodiments of an electrode pad assembly in which a handle is provided for the rescuer; and

FIG. 10 is a drawing of a CPR assistance device in accordance with an embodiment of the present invention.

### DETAILED DESCRIPTION

Aspects and embodiments of the present invention are not limited to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” “having,” “containing,” “involving,” and variations thereof herein is meant to encompass the items listed thereafter and equivalents thereof.

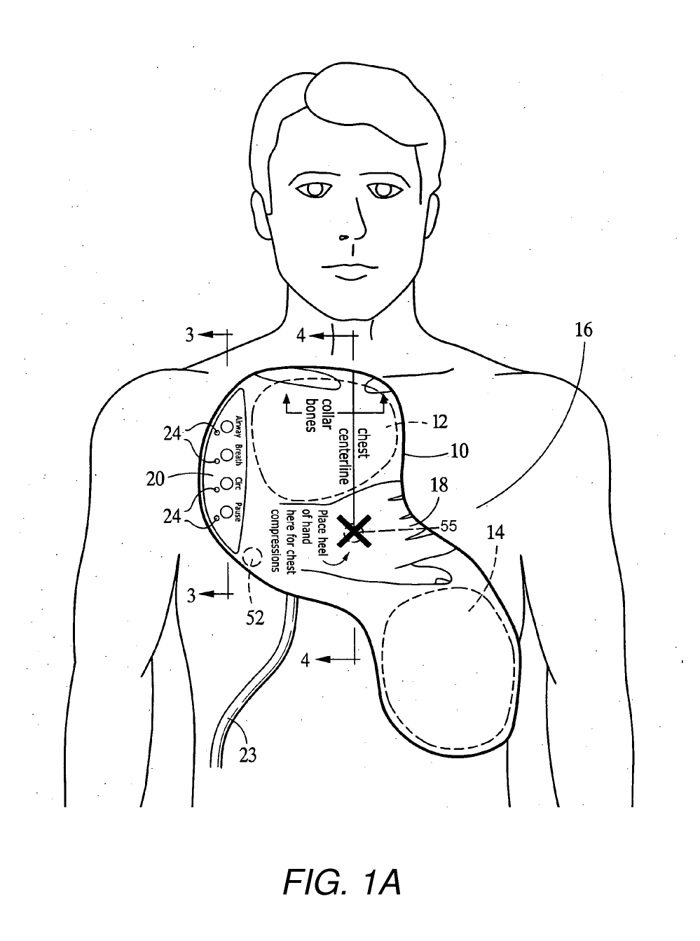

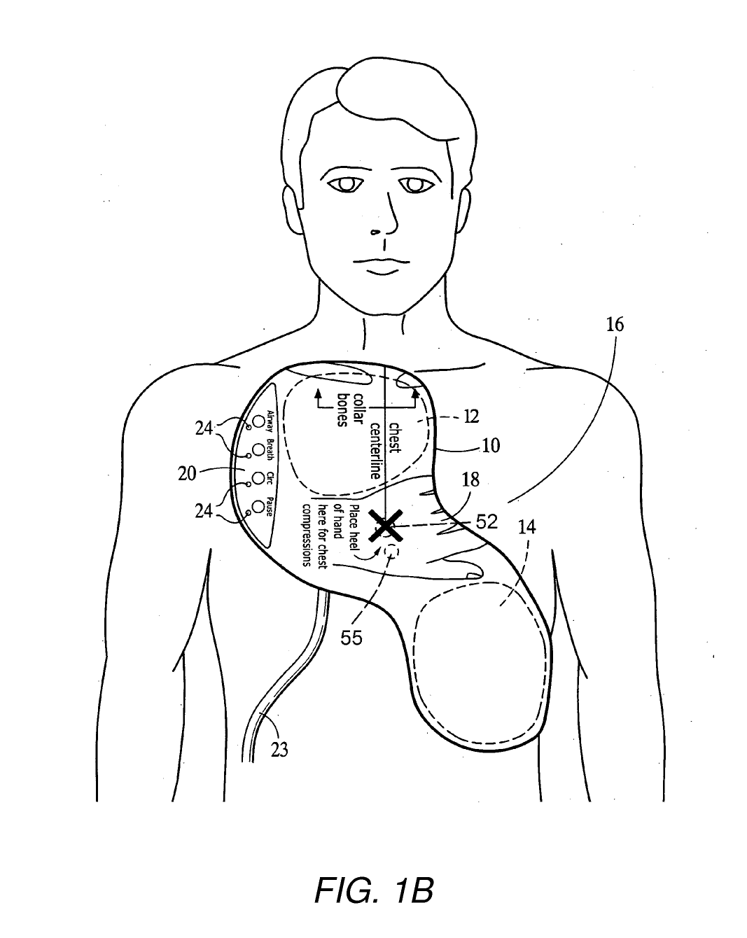

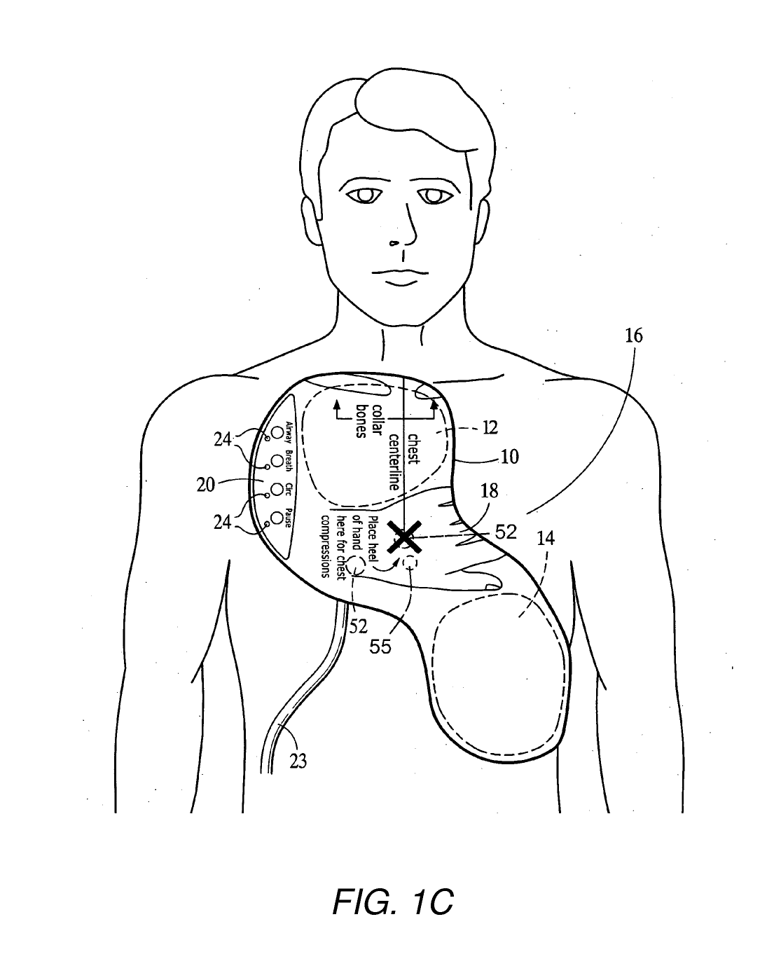

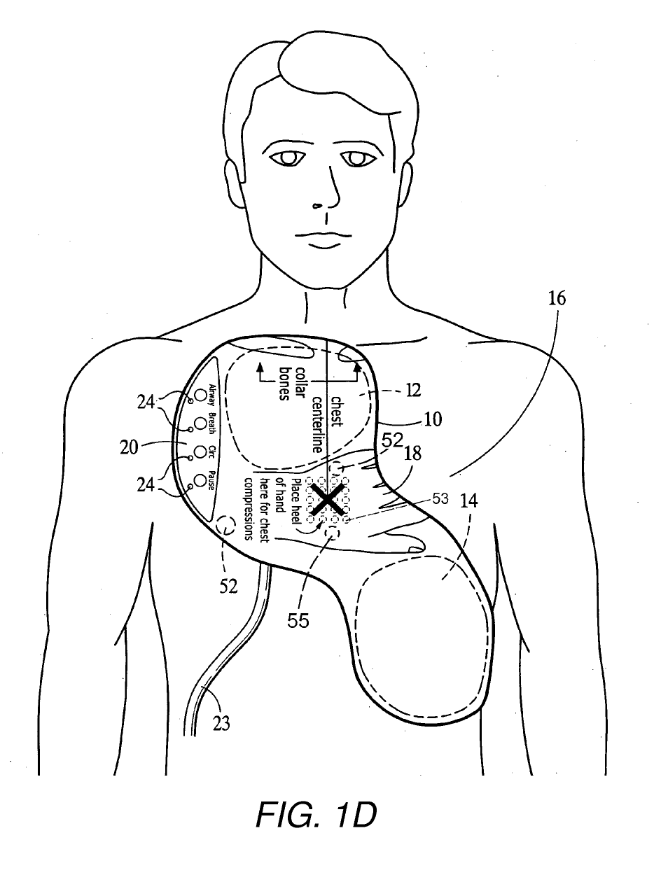

FIG. 1A illustrates a defibrillation electrode pad 10 similar to those described in commonly owned Application Publication No. US 2005/0131465, which is incorporated herein by reference in its entirety. The defibrillation electrode pad 10 includes a high-voltage apex defibrillation electrode 12 and a high-voltage sternum defibrillation electrode 14. The defibrillation electrode pad 10 is placed on the subject's chest 16 and includes a region 18 on which a user may press to perform CPR. Legends on pad 10 indicate proper placement of the pad with respect to the subject's collarbones and the chest centerline and the proper placement of the heel of the rescuer's hand. In some embodiments, the electrodes 12, 14 are also used to to monitor an electrocardiogram (ECG) signal of a subject.

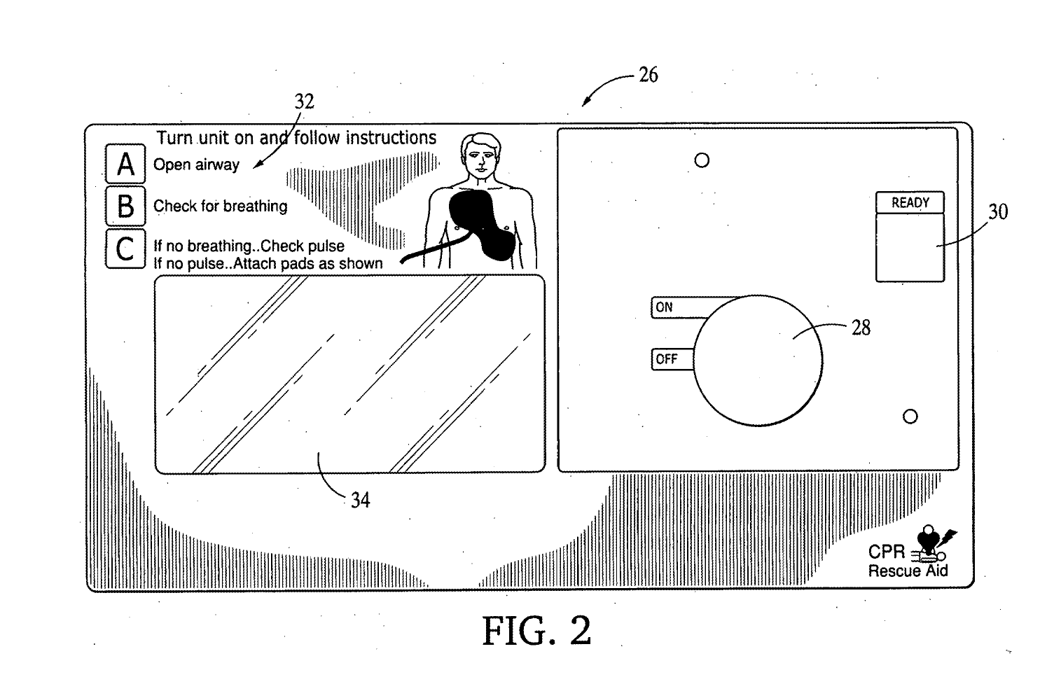

A low-profile button panel 20 is provided on the electrode assembly. Button panel 20 has buttons, including buttons labeled “Airway” (Airway Help), “Breath” (Breathing Help), “Circ” (Circulation Help) and “Pause,” and may also include adjacent light emitting diodes (LEDs) 24 that indicate which button has been most recently pressed. Button panel 20 is connected by a cable 23 to a remote resuscitation control box 26, shown in FIG. 2. Button panel 20 provides rigid support underneath buttons “Airway,” “Breath,” “Circ,” and “Pause” against which switches included in the buttons can be pushed in order to ensure good switch closure while the electrode pad 10 rests on a subject. Button panel 20 includes components that make electrical contact with silver/silver-chloride electrical circuit components screen-printed on a polyester base of defibrillation electrode pad 10, as is described in detail below.

A pulse detection system 52 based on shining light through the subject's vascular bed, for example, a pulse oximetry system, is incorporated into defibrillation electrode pad 10. In the defibrillation electrode pad of FIG. 1A, the pulse detection system 52 is positioned on the side of the defibrillation electrode pad 10 which, in use, is disposed against the subject. In accordance with an aspect of the present invention, rather than positioning the pulse detection system 52 in a location displaced from a position where compression is applied during CPR (for example, the region denoted by the “X” in FIG. 1A), the pulse detection system 52 may instead be positioned proximate or beneath an area of the electrode pad 10 to which a rescuer may apply pressure while administering CPR to the subject (FIG. 1B). In accordance with an aspect of the present invention, where the pulse detection system is positioned proximate the area to which compressions are applied, the pulse detection system 52 may be utilized to measure perfusion of the subject as well as the deperfusion and reperfusion of an area of the subject's chest that occurs due to the compressions on the skin surface during the administration of CPR. The measurements of deperfusion and/or reperfusion may be utilized to identify the effectiveness of the CPR being performed. For example, if there is less than a certain amount of deperfusion during the compression portion of CPR administration, this may be indicative of too little pressure being applied during the compression portion. Under such conditions, the to defibrillation electrode pad 10 may be programmed to provide instructions to a rescuer to increase the pressure and/or depth of chest compressions. Further, a high rate of reperfusion during a relaxation portion of CPR administration may be indicative of return of spontaneous circulation (ROSC) in the subject. Under such conditions, the defibrillation electrode pad 10 (or electronic components associated with the defibrillation electrode pad 10) may provide instructions to a rescuer to check the subject for signs of a pulse and/or to terminate the administration of CPR. In some embodiments, the defibrillation electrode pad 10 (or electronic components associated with the defibrillation electrode pad 10) may periodically, for example, once about every 12 compressions, instruct the rescuer to halt CPR to provide sufficient time, for example, about two seconds, for the pulse detection system to take a substantially noise-free reading of the perfusion of the subject.

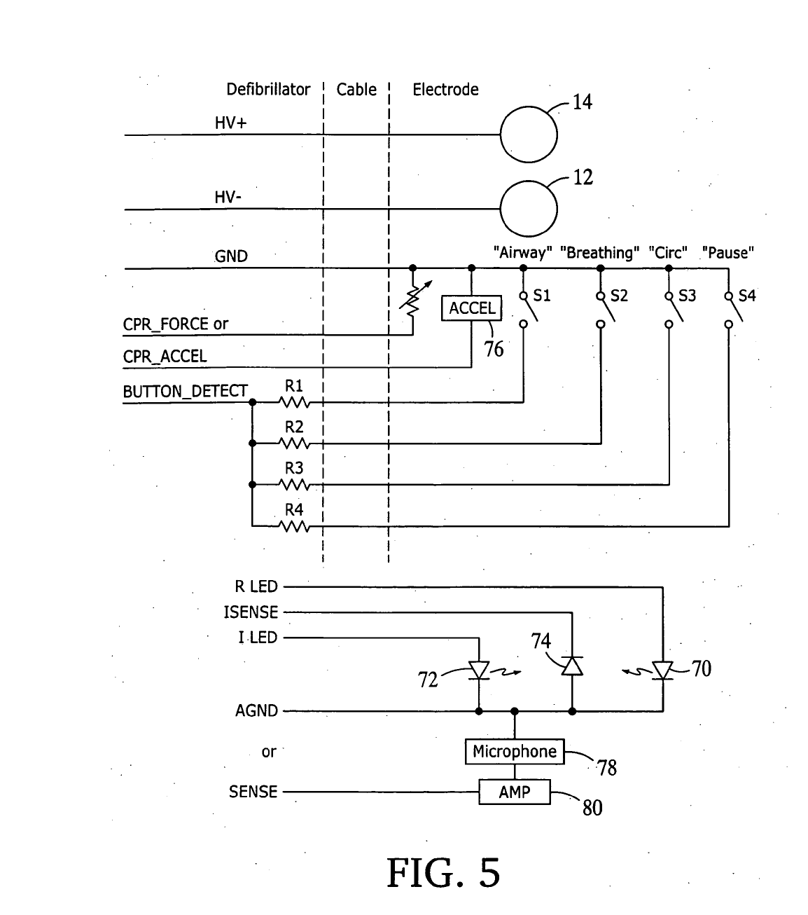

In some embodiments, the pulse detection system 52 comprises a pulse oximetry system which includes a red light-emitting diode 70, a near-infrared light-emitting diode 72, and a photodetector diode 74 (see FIG. 5) incorporated into defibrillation electrode pad 10 in a manner so as to contact the surface of the subject's chest 16. The red and near-infrared light-emitting diodes 70, 72 emit light at two different wavelengths, which is diffusely scattered through the subject's tissue and detected by the photodetector diode 74. The information obtained from the photodetector diode 74 can be used to determine whether the subject's blood is oxygenated, according to known noninvasive optical monitoring techniques.

In some embodiments, a pulse detection system 52 may include a pulse oximetry system including multiple sensors, each including light-emitting diodes and photodetector diodes, which monitor blood perfusion in different areas of a subject's chest. A first sensor may be placed proximate the center of the defibrillation electrode pad 10, for example, proximate or beneath the “X” illustrated in FIG. 1C, and one or more other sensors could be placed proximate a periphery of the compression area of the defibrillation electrode pad 10, for example, proximate a periphery of region 18, and/or proximate a periphery of the defibrillation electrode pad 10. The use of a pulse oximetry system including multiple sensors can provide information regarding how to quickly blood leaves and returns to the chest area in the zone of compression of the subject during CPR during compression and/or relaxation phases of the CPR administration, to provide an indication of the motion of blood from one area to another beneath the subject's skin, and/or to provide a “map” of the degree of blood perfusion across a selected area beneath the subject's skin.

In another embodiment, the pulse detection system 52 may include an impedance measurement system. As blood enters a region beneath a subject's skin, the impedance of the subject's skin in that region may decrease. A measurement of the impedance, or a measurement of a change in impedance across a portion of a subject's skin, may thus provide an indication of whether blood is entering and/or exiting from the region beneath the measured portion of the subject's skin. The impedance measurement system may include at least two electrodes 53 configured and arranged to contact the subject's skin. The electrodes may be positioned on a surface of the side of the defibrillation electrode pad 10 which, in use, is disposed against the subject and may be configured to make electrical contact with the surface of the skin of the subject. Monitoring and control electronics associated with the defibrillation electrode pad 10, described below, may include circuitry in communication with the electrodes to measure and analyze the impedance of the subject's skin. In some embodiments, three or more electrodes may be utilized in the impedance measurement system. The electrodes may be arranged in a grid-like formation as illustrated in FIG. 1D. The use of multiple electrodes may allow for the impedance measurement system to provide an indication of the motion of blood from one area to another beneath the subject's skin and/or to provide a “map” of the degree of blood perfusion across a selected area beneath the subject's skin. The multiple electrodes may be arranged in an array, for example, a grid-like array, and may form an electric field sensor or electric field imaging device capable of monitoring perfusion of the subject. Electric field sensors are well known and operate in accordance with the principals described in, for example, Joshua Smith et al., Electric Field Sensing for Graphical Interfaces, Computer Graphics and Applications, Vol. 18, No. 3, 54-61, (1998) and Joseph A. Paradiso et al., Musical Applications of Electric Field Sensing, Computer Music Journal, Vol. 21, No. 269-89 (1997). An to example of a commercially available electric field imaging device is the model number 33794 electric field imaging device available from Freescale Semiconductor, Inc.

In another embodiment, the pulse detection system 52 includes a phonocardiogram system for listening to the sound of the subject's heart in addition to or instead of a pulse oximetry and/or impedance measurement system. The phonocardiogram system may include a microphone and an amplifier incorporated within the electrode pad. Because a heart sound can be confused with microphone noise, the signal processing that must be performed by the microprocessor inside the control box will be more difficult in connection with a phonocardiogram system than in connection with a pulse oximetry system. Nevertheless, there are programs available that can enable the microprocessor to determine whether an ECG signal is present as opposed to microphone noise.

Pulse oximetry is a well-developed, established technology, but it requires good contact between the light sources and the subject's skin so that light can shine down into the subject's vascular bed. Many subjects may have a significant amount of chest hair, which can interfere with good contact. It may be desirable for different types of electrode pads to be available at a given location (one having a pulse oximetry system and one having a phonocardiogram system) so that a rescuer can select an appropriate electrode pad depending on the nature of the subject.

In some embodiments, a pulse detection system as described above may be utilized in systems other than a defibrillation electrode pad 10. For example, a pulse detection system as described above may be provided in a system that does not include defibrillation electrodes. Such systems include CPR assistance devices, for example, the PocketCPR® CPR assistance device available from ZOLL Medical Corp. Examples of CPR assistance devices are disclosed in U.S. Pat. Nos. 7,245,974, 8,096,962, 6,390,996, 6,782,293, 7,108,665, 7,118,542, and 7,122,014.

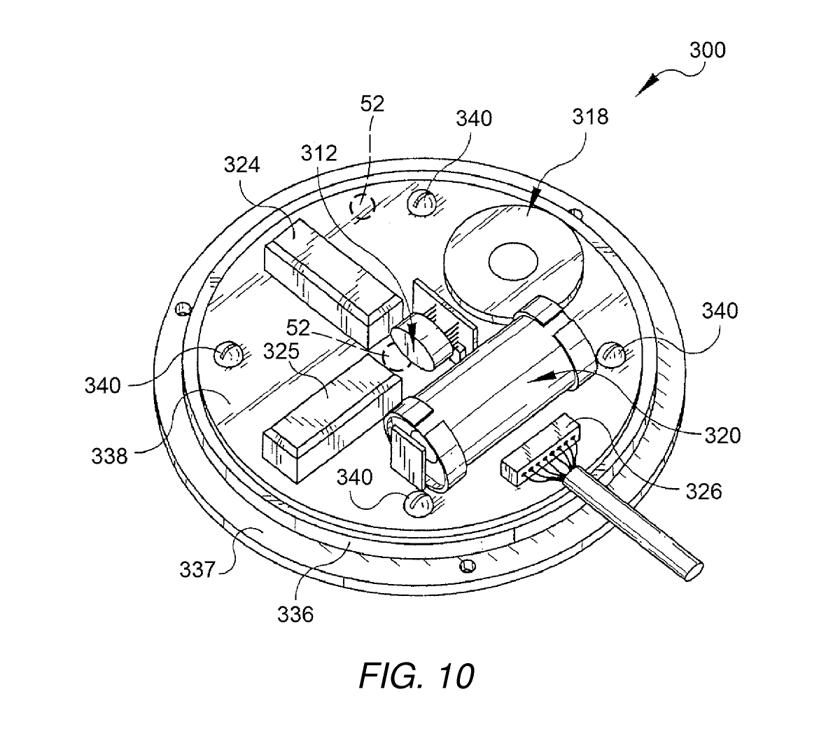

One particular example of a CPR assistance device 300 is illustrated in FIG. 10 in perspective view with the cover removed to reveal the internal components. The CPR assistance device 300 comprises a circular base 336 having an outer flange portion 337. Mounted on base 336 is a circuit board 338. Circuit board 338 is fixed to base 336 by fasteners 340. The CPR assistance device 300 includes a displacement detector 312 such as an accelerometer coupled to an external microprocessor via an interface 326. The CPR assistance device 300 includes a signaling mechanism comprising an audible indicator (i.e., a speaker) 318, which has an input connected to the microprocessor via interface 326. A DC voltage power supply 320 is included in the body of the CPR assistance device. The DC voltage power supply 320 provides a DC voltage for powering the various components of the illustrated CPR assistance device 300, including the above-noted displacement detector 312 and audible indicator 318. Tilt compensation devices are provided which include a first gyroscope 324 and a second gyroscope 325. The first gyroscope 324 and second gyroscope 325 each include outputs connected to the microprocessor via interface 326. The CPR assistance device 300 may also include one or more pulse detection systems 52. Where multiple pulse detection systems are used, one may be disposed in a central region of the CPR assistance device, and another disposed in a peripheral region as shown in FIG. 10.

In another example, a pulse detection system as described herein may be included in a chest compression belt such as is disclosed in U.S. Pat. No. 7,131,953, titled CPR ASSIST DEVICE ADAPTED FOR ANTERIOR/POSTERIOR COMPRESSIONS, issued Nov. 7, 2006, which is incorporated herein by reference in its entirety. Pulse detection systems as described above may be used in any of various treatment systems and are not limited to use in the embodiments disclosed herein.

In another embodiment, instead of providing a low-profile button panel, a button housing can be provided that is affixed to an edge of the defibrillation electrode. The housing may be in the form of a clamshell formed of single molded plastic element having a hinge at an edge of the clamshell around which the plastic bends. The two halves of the clamshell can be snapped together around the electrode assembly.

The resuscitation control box (FIG. 2) includes an internal charge storage capacitor and associated circuitry including a microprocessor, and further includes off/on dial 28, and a “READY” button 30 that the rescuer presses immediately prior to to application of a defibrillation shock in order to ensure that the rescuer is not in physical contact with the subject. The microprocessor may be a RISC processor such as a Hitachi SH-3, which can interface well with displays and keyboards, or more generally a processor capable of handling DSP-type (digital signal processing) operations.

The resuscitation control box has printed instructions 32 on its front face listing the basic steps A, B, and C for resuscitating a subject and giving basic instructions for positioning the defibrillation electrode pad on the subject. A speaker included in the resuscitation control box orally prompts the user to perform various steps, as is described in detail below.

For example, the resuscitation control box instructs the user, by audible instructions and/or through a display 34 on the resuscitation control box, to check the subject's airway and perform mouth-to-mouth resuscitation, and if the subject's airway is still blocked, to press the “Airway” (Airway Help) button on the button panel (FIG. 1A), upon which the resuscitation control box gives detailed prompts for clearing the subject's airway. If the subject's airway is clear and the subject has a pulse but the subject does not breathe after initial mouth-to-mouth resuscitation, the resuscitation control box instructs the user press the “Breath” (Breathing Help) button, upon which the resuscitation control box gives detailed mouth-to-mouth resuscitation prompts. If, during the detailed mouth-to-mouth resuscitation procedure, the rescuer checks the subject's pulse and discovers that the subject has no pulse, the resuscitation control box instructs the user to press the “Circ” (Circulation Help) button.

During the circulation procedure, the resuscitation control box receives electrical signals from the defibrillation electrodes and determines whether defibrillation or CPR should be performed. If the resuscitation control box determines that defibrillation is desirable, the resuscitation control box instructs the user to press the “READY” button on the resuscitation control box and to stand clear of the subject. After a short pause, the resuscitation control box causes a defibrillation pulse to be applied between the electrodes. If at any point the resuscitation control box determines, based on the electrical signals received from the electrodes, that CPR is desirable, it will instruct the user to perform CPR.

In the described embodiment, the controls for the system may be disposed on the electrodes attached to the subject rather than the resuscitation control box. This is important because it enables the rescuer to remain focused on the subject rather than the control box. The resuscitation control box gets its information directly from the electrodes and the controls on the electrodes.

The resuscitation control box can sense electrical signals from the subject's body during pauses between CPR compressions. Also, as is described below, a compression or displacement-sensing element 55 as shown in FIGS. 1A-1D, such as an accelerometer or a force-sensing element, may be provided in the region of the defibrillation electrode pad on which the user presses to perform CPR. The purpose of the compression or displacement-sensing or force-sensing element 55 is to allow the resuscitation control box to prompt the user to apply additional compression or force, to vary the depth or rate of chest compressions, or to prompt the user to cease CPR if the user is performing CPR at an inappropriate point in time.

Referring to FIG. 4, in one embodiment, each electrode 12, 14 (only electrode 12 is shown) of defibrillation electrode pad 10 includes a polymer-based ink containing a silver/silver-chloride suspension, which is screen-printed on a polyester or plastic base 36.

The ink is used to carry the defibrillation current. The screen-printing process first involves applying a resist layer to the polyester base 36. The resist layer is basically a loose mesh of nylon or the like, in which the holes have been filled in at some locations in the mesh. Then, the silver/silver-chloride ink is applied as a paste through the resist layer in a squeegee-like manner. The ink squeezes through the screen and becomes a solid layer. The ink may then be cured or dried. The silver/silver-chloride ink provides good conductivity and good monitoring capabilities.

Thus, the ink can be applied as pattern, as opposed to a solid sheet covering the entire polyester base. For example, U.S. Pat. No. 5,330,526 describes an electrode in which the conductive portion has a scalloped or daisy shape that increases the circumference of the conductive portion and reduces burning of the subject. A conductive adhesive gel 38 covers the exposed surface of each electrode.

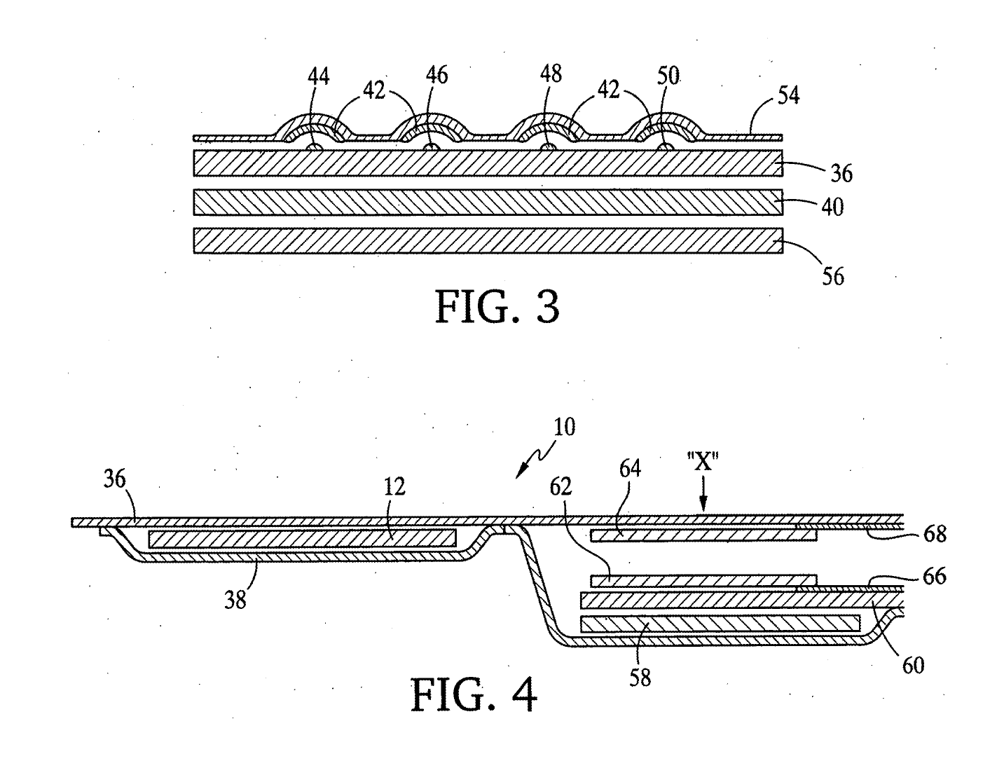

In addition, electrical circuit components may also be screen printed on the base, in the same manner as flat circuit components of membrane-covered, laminated panel controls. Referring to FIG. 3, a rigid piece 40 of hard plastic, such as PVC or polycarbonate, is laminated beneath substrate 36 and supports buttons “Airway,” “Breath,” “Circ,” and “Pause.” The rigid plastic piece 40 is glued onto substrate 36. Buttons “Airway,” “Breath,” “Circ,” and “Pause” consist of small metal dome snap-action switches that make contact between an upper conductive ink trace 42 and lower conductive ink traces 44, 46, 48, and 50. Buttons “Airway,” “Breath,” “Circ,” and “Pause” serve as controls that can be activated by the user that are physically located either on or immediately adjacent to the electrode assembly itself. Each of buttons “Airway,” “Breath,” “Circ,” and “Pause” may be associated with an adjacent light-emitting diode (LED). For example, LEDs may be glued, using conductive epoxy, onto silver/silver-chloride traces on substrate 36. An embossed polyester laminate layer 54 covers conductive ink trace 42 of buttons “Airway,” “Breath,” “Circ,” and “Pause,” and a foam layer 56 is laminated beneath rigid plastic piece 40.

Referring again to FIG. 4, defibrillation electrode pad 10 includes an extension piece that is placed directly over the location on the subject's body where the rescuer performs chest compressions. This extension piece includes substrate 36, and a semi-rigid plastic supporting member 58 laminated underneath substrate 36 that covers the chest compression area. Semi-rigid supporting member 58 provides somewhat less rigidity than rigid plastic piece 40 provided at the location of buttons “Airway,” “Breath,” “Circ,” and “Pause” (illustrated in FIG. 3).

In embodiments having a force-sensing element, a polyester laminate 60, and a force-sensing resistor having two layers of carbon-plated material 62 and 64, are laminated between polyester substrate 36 and semi-rigid supporting member 58. A suitable construction of the force-sensing resistor is illustrated in the FSR Integration Guide & Evaluation Parts Catalog with Suggested Electrical Interfaces, from Interlink Electronics. The electrical contact between the two carbon-plated layers of material increases with increased pressure, and the layers of force-sensing resistive material can provide a generally linear relationship between resistance and force. Conductive ink traces 66 and 68 provide electrical connections to the two layers of the force-to sensing resistor.

During chest compressions, the rescuer's hands are placed over the extension piece, and the force-sensing resistor of the extension piece is used to sense the force and the timing of the chest compressions. The force-sensing resistor provides information to the resuscitation control box so that the resuscitation control box can provide the rescuer with feedback if the rescuer is applying insufficient force. The resuscitation control box also provides coaching as to the rate at which CPR is performed. In certain situations, the resuscitation control box indicates to the rescuer that CPR should be halted because it is being performed at an inappropriate time, such as immediately prior to application of a defibrillation shock when the rescuer's hands should not be touching the subject, in which case the resuscitation control box will also indicate that the rescuer should stay clear of the subject because the subject is going to experience a defibrillation shock.

As is noted above, during CPR the rescuer pushes on the subject's chest through the extension piece in the vicinity of the electrodes 12, 14. If the resuscitation control box were to perform analysis during the chest compressions, the chest compressions would be likely to affect the sensed electrical rhythm. Instead, during pauses between sets of compressions (for example, during a pause after every fifth chest compression), the resuscitation control box can perform an electrocardiogram (ECG) analysis. The resuscitation control box might discover, for example, that a subject who is undergoing CPR and was exhibiting a non-shockable rhythm such as bradycardia is now exhibiting ventricular fibrillation. In such an instance, the resuscitation control box would instruct the rescuer to stop performing CPR so as to allow the resuscitation control box to perform more analysis and possibly apply one or more shocks to the subject. Thus, the rescuer is integrated into a sophisticated scheme that allows complex combinations of therapy.

In other embodiments, a compression-sensing element such as an accelerometer 55 may be used in addition to or in place of a force-sensing element. In some embodiments, the accelerometer, such as a solid-state ADXL202 accelerometer, is positioned at the location where the rescuer performs chest compressions, for example, proximate or beneath the “X” illustrated in FIGS. 1A-1D. In these embodiments, the microprocessor obtains acceleration readings from the accelerometer 55 at fixed time intervals such as one-millisecond intervals, and the microprocessor integrates the acceleration readings to provide a measurement of chest compression. The use of an accelerometer is based on the appreciation that it may be more important to measure how deeply the rescuer is compressing the chest than to measure how hard the rescuer is pressing. Every subject's chest will have a different compliance, and it has been found that a subject's chest should be compressed about an inch and a half to two inches in a normal sized adult regardless of the subject's chest compliance for CPR to be most effective.

In some embodiments, a signal from a displacement monitor, for example, the force-sensing element and/or accelerometer 55 described above may be utilized to time measurements performed by the pulse detection system. Monitoring and control electronics in, for example, the resuscitation control box may perform a measurement of blood perfusion of an area of the subject's chest (or other body region) responsive to a signal from the displacement monitor indicative of a desired portion of a CPR administration cycle being achieved. For example, a blood perfusion measurement may be taken by the pulse detection system 52 when the displacement monitor indicates that the chest of the subject is in a fully relaxed portion of a CPR cycle. The pulse detection system may be utilized to determine if the subject has experienced the return of pulsatile perfusion while the chest is in a relaxed state. In some embodiments, the rescuer may be prompted by the system, for example, by the resuscitation control box, to suspend CPR for a time period of, for example, about two seconds while the pulse detection system determines if the subject has experienced the return of pulsatile perfusion. Based on the measurement by the pulse detection system, the rescuer may be prompted by the system, for example, by the resuscitation control box, to resume CPR and/or, for example, if pulsatile perfusion is not detected, or if some pulsatile perfusion were detected, to take a pulse measurement. By providing prompts to the rescuer to take a pulse measurement only when there is an indication that one may be present, for example, due to the presence of an indication of pulsatile perfusion, unnecessary and potentially harmful cessations of CPR to check for a pulse may be avoided when it is unlikely that one is present.

A blood perfusion measurement may additionally or alternatively be taken by the pulse detection system 52 when the displacement monitor indicates that the chest of the subject is in a fully compressed (apex) portion of a CPR cycle. The difference in blood perfusion under the compressed and relaxed conditions of the subject's chest may provide useful information regarding the degree to which the subject's heart has begun to beat on its own and/or of the onset of the return of spontaneous circulation (ROSC).

In other embodiments, the pulse detection measurement using the pulse detection system 52 and/or impedance measuring electrodes may be initiated responsive to the displacement monitor providing a signal indicative of a state of compression of a subject's chest at one or more intermediate points between a fully compressed state and a fully relaxed state. The pulse detection measurement may also be taken during the time that a compression is being performed or during a time in which a relaxation portion of a CPR cycle being performed as indicated by a signal from the displacement monitor. Further, the pulse detection measurement can be used as additional feedback to a rescuer regarding the quality of the CPR being performed and may be used to modify instructions provided to the rescuer regarding the performance of the CPR.

Where multiple pulse detection systems 52 are used, a difference in perfusion between different areas of the subject and/or a rate of flow of blood from one area of the subject's body to another, for example, from one region of the subject's chest to another, may be monitored. The difference in perfusion between different areas of the subject and/or a rate of flow of blood from one area of the subject's body to another may be analyzed and may provide information regarding the effectiveness of the CPR being performed, the degree to which the subject's heart has begun to beat on its own, and/or of the onset of ROSC.

A displacement monitor may be utilized in conjunction with a pulse detection system as described above in any of a defibrillation electrode pad 10, a mechanically assisted chest compression device, or any other CPR assistance or treatment device.

FIG. 5 is a schematic diagram illustrating the circuit interconnections between the defibrillation electrode pad of any of FIGS. 1A-1D through the cable 23 to the to resuscitation control box of FIG. 2. Sternum electrode 14 is connected to HV+ at the resuscitation control box, and apex electrode 12 is connected to HV−. A ground GND is connected to the upper conductive ink trace of buttons “Airway,” “Breath” (labeled as “Breathing” in FIG. 5), “Circ,” and “Pause” and to one of the layers of the force-sensing resistor.

The other layer of the force-sensing resistor is connected to CPR_FORCE, and the lower conductive ink traces associated with buttons “Airway,” “Breath” (labeled as “Breathing” in FIG. 5), “Circ,” and “Pause” are connected to BUTTON_DETECT through resistors R1, R2, R3, and R4. In addition to, or as an alternative to the use of a force-sensing resistor, a compression-sensing accelerometer 76 may be employed, in which case CPR_FORCE is augmented or replaced by CPR_ACCEL connected to accelerometer 76. Red light-emitting diode 70, near-infrared light-emitting diode 72, and photodetector diode 74 of the pulse oximetry system are connected to RLED, ILED, and ISENSE respectively, as well as ground AGND. In addition to, or as an alternative to the use of a pulse oximetry system, a phonocardiogram system may be employed, in which case RLED, ILED, and ISENSE are augmented or replaced by SENSE connected to microphone 78 and amplifier 80.

The combined defibrillation and CPR resuscitation assembly provided can be less intimidating than conventional AEDs because the assembly is not devoted solely to defibrillation. Moreover, the resuscitation assembly is less intimidating because it accommodates common skill retention problems with respect to necessary techniques ancillary to defibrillation such as mouth-to-mouth resuscitation and CPR, including the appropriate rates of chest compression, the proper location for performing compressions, and/or the proper manner of tilting the subject's head. In addition, because the rescuer knows that it may never even be necessary to apply a defibrillation shock during use of the resuscitation assembly, the rescuer may be more comfortable using the resuscitation assembly for mouth-to-mouth resuscitation and CPR. Unlike previous CPR prompting devices, the rescuer would be required to place the electrode assembly on top of the subject, but the rescuer would do this with the belief that the resuscitation assembly will be sensing the subject's condition and that the likelihood that the resuscitation assembly is actually going to apply a shock is low.

If, during a resuscitation process, the resuscitation control box instructs the rescuer to press the “READY” button so that a defibrillation shock can be applied, the rescuer will likely feel comfortable allowing the shock to be applied to the subject. The resuscitation assembly tells the rescuer what to do, and, given that the rescuer is already using the assembly, the rescuer is likely to do what the rescuer is told to do. The rescuer will be likely to view the resuscitation assembly as being a sophisticated CPR prompting device with an additional feature incorporated into it, and since rescuers are less likely to be intimidated by CPR prompting devices than AEDs, they will be likely to use the resuscitation assembly when it is needed.

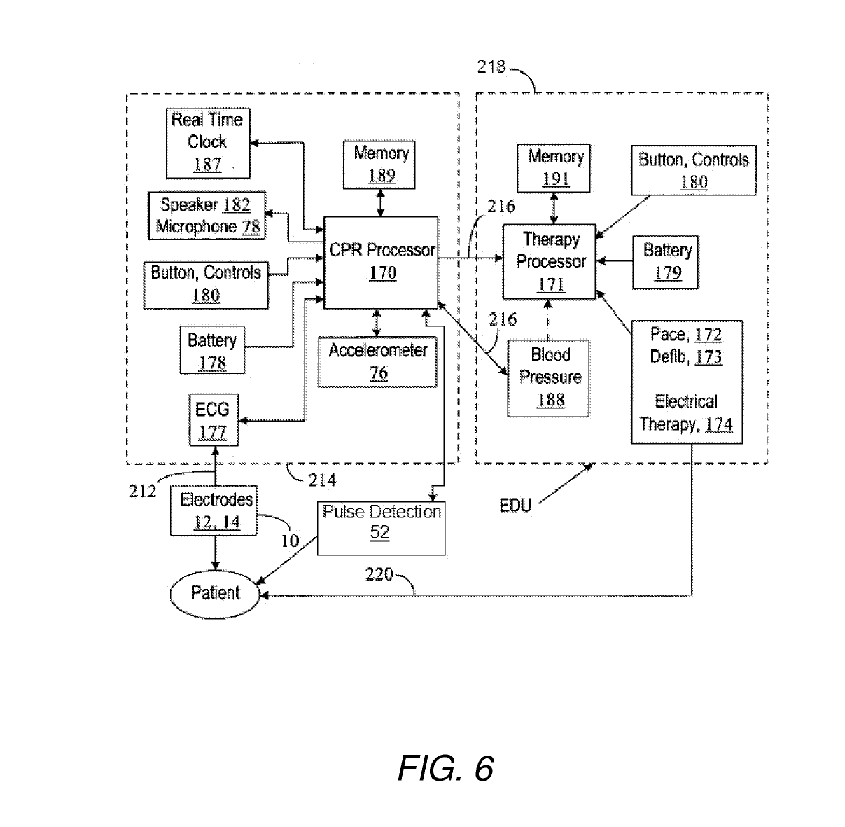

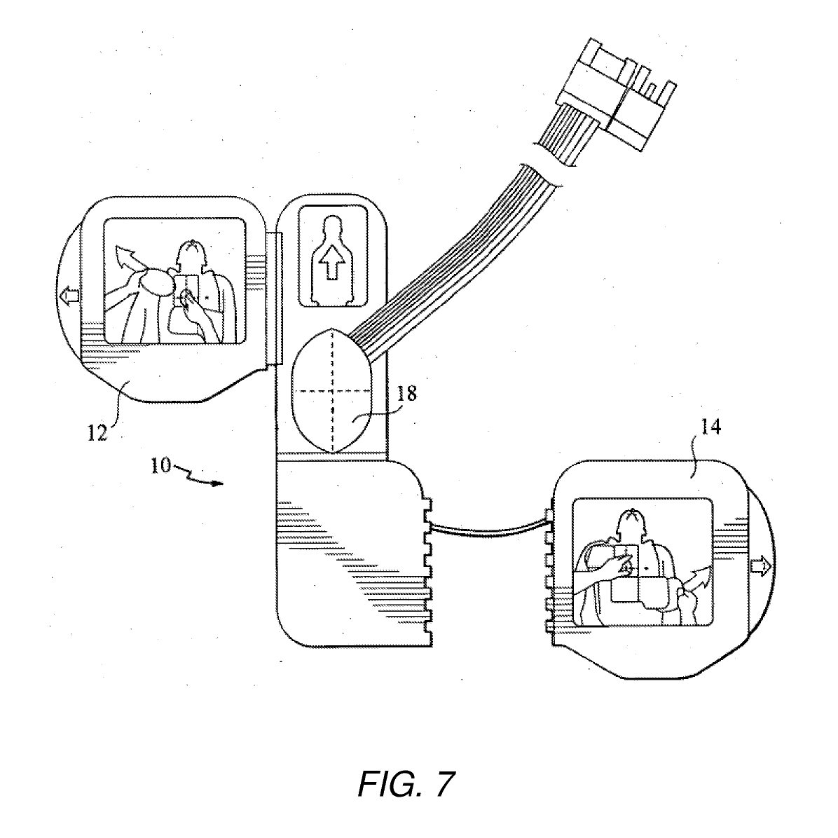

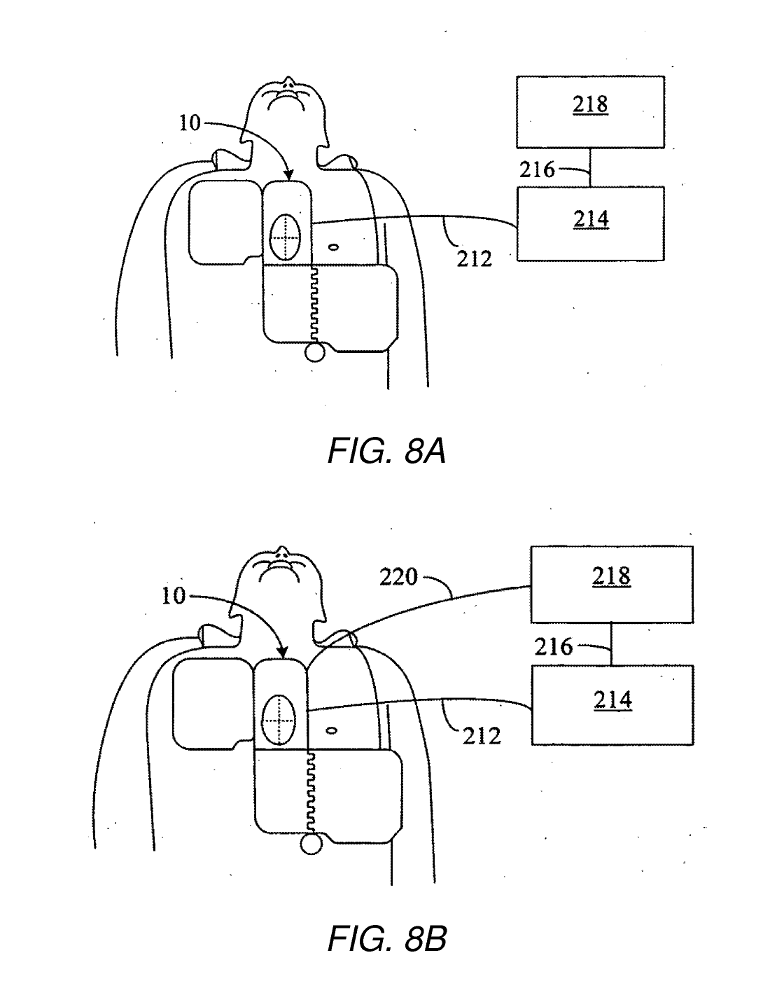

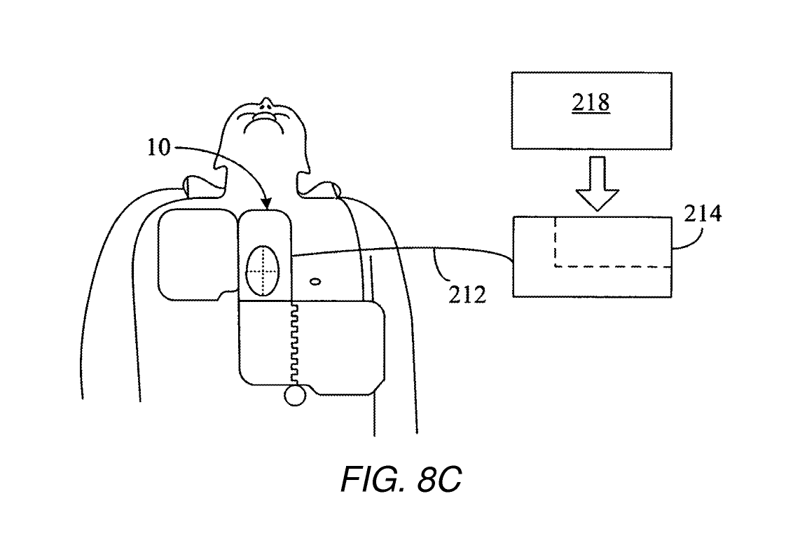

FIGS. 6, 7, and 8A-8C show alternative embodiments in which an electrode pad assembly 10 is connected by a cable 212 to a first unit 214 containing the electronics for CPR prompting and resuscitation control. Another cable 216 connects the first unit to a second unit 218 containing the electronics for defibrillation and pacing therapy. A third cable 220 could be provided for making a direct connection from the second unit to the electrodes (FIG. 8B). The first unit 214 could be configured to receive the second unit 218 as an inserted module (FIG. 8C), in which case the electrical connection between the units are made internally without the use of cable 216. The primary function of the first unit 214 is to provide processing and control for CPR functions such as CPR prompts. The primary function of the second unit 218 is to provide processing and control of electrical therapy functions. The first unit includes a CPR processor 170, a battery 178, ECG circuitry 177 for amplifying and filtering the ECG signal obtained from the defibrillation pads 12, 14, a microphone 78 for recording the rescuer's voice as well as ambient sounds, an accelerometer 76, a real time clock 187, and a speaker 182 for delivering prompts to the rescuer. As shown, the first unit may further include at least one pulse detection unit 52. The CPR processor 170 may be configured to receive input from the accelerometer 76, and based on that input, to instruct the at least one pulse detection system 52 to make a perfusion measurement based on whether the subject's chest is in a compressed state, a relaxed state, in an intermediate state, or during compression or relaxation of the subject's chest as desired. The second unit includes a therapy to processor 171, a battery 179, buttons and controls 180, and memory 191.

The first unit could also be incorporated into the electrode pad assembly rather than being a separate box. The electronics could be provided on the rigid substrate 40 of the electrode pad assembly (FIG. 1).

Separate batteries 178, 179 and controls 180 may be provided for the first (CPR) and second (therapy) units, thereby allowing the electronics in the first unit to provide CPR prompting to the operator without the need for the second unit. The cable 216 that connects the first and second units may be detachable. Memory 189 is provided in the first unit for storing information such as voice recording, ECG data, chest compression data, perfusion data, or electronic system status such as device failures that occur during daily self checks of the electronics initiated by a real time clock circuit.

The defibrillation electrode pad assembly 10 may incorporate defibrillation electrodes composed of a material that can be held against a subject's skin for extended periods of time (for example, up to 30 days).

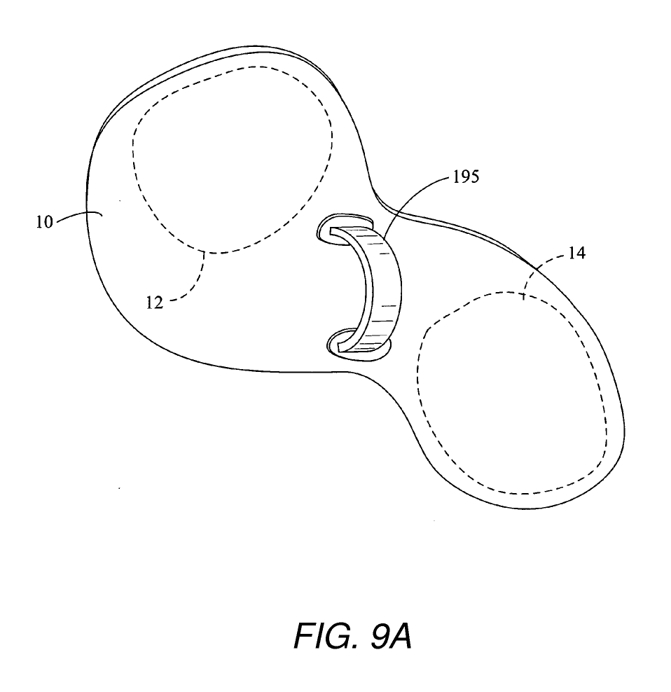

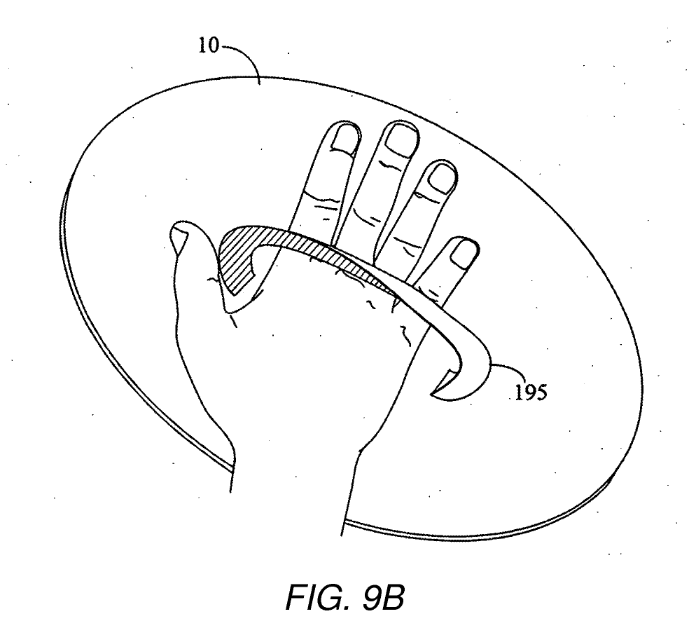

As shown in FIGS. 9A and 9B, the pad assembly 10 may also incorporate features on its upper surface facing the rescuer that provide a handle 195 for the rescuer during performance of CPR. The handle could take the form of a fabric loop (FIG. 9B) or a more rigid polymer member (FIG. 9A). The fabric could be sewn or adhered by adhesive or ultrasonic bonding to the pad 10 (FIG. 9B). The polymer handle could also be bonded by adhesive or ultrasonic bonding to the pad (FIG. 9A). It has been shown in studies that the maintenance of pressure on the chest during the decompression phase of chest compression results in a significant decrease in the effectiveness of the chest compressions. The handle 195 motivates the rescuer to pull up at least slightly during the decompression phase. The adhesive gel of the electrode pad, or other adhesive, can extend under the region where the rescuer's hands are placed during compression thus providing adhesion of the pad to the skin while the rescuer pulls on the handle during the decompression phase. Pulling up on the chest during the decompression phase has been shown to heighten negative intrathoracic pressure, increasing venous return and thus increasing blood flow during chest compressions.

In another embodiment, the first unit may be adapted to be supported by the subject for long periods of time. The unit could be incorporated into the electrode pad assembly as suggested above, or it could be a separate unit configured to be worn by the subject. In such an embodiment, the electronics of the first unit are designed to allow for long term monitoring of the subject's condition via the ECG 177 and/or other physiological monitoring circuitry. If a physiological condition is detected that is deemed hazardous to the subject by the CPR processor 170, based on analysis of the ECG and/or other physiological parameters, an alarm is sounded to the subject via the speaker 182.

An activity sensor and associated circuitry can inform the CPR processor of whether the subject is moving. For example, accelerometer 76 could serve as the activity sensor, and detect whether or not the subject is moving. Subject motion may be detected using a variety of different algorithms, including, for example, the following: the acceleration signal is integrated over one-second intervals to provide an estimate of velocity; velocity is integrated over the same one-second intervals to provide an estimate of displacement; the root means square velocity is calculated for each one-second interval; if either the RMS velocity exceeds a predetermined value, for example, about 0.2 cm/s or the peak displacement exceeds a predetermined value, for example, about 0.5 cm, the subject is determined to be moving.

If the algorithm determines that a cardiac emergency event is occurring, the first unit can send a message directly to a medical emergency response system, such as 911. This can be done using a variety of known communication techniques, for example, Bluetooth, cellular phone, or Ultra Wideband (UWB). If the activity sensor has determined that the subject is still moving during the cardiac emergency, the unit could also issue a prompt indicating, for example, “Call 911 Immediately!”

The first unit will be able to determine the orientation of the subject, for example, based on a signal output from the accelerometer. It can detect if a subject has fallen down and initiate a message to the emergency system. It can also determine whether the subject is lying on their back; the proper orientation for doing CPR. Thus, a specific prompt can be provided to the rescuer that tells them to roll the subject on their back prior to beginning CPR, should the device detect an improper orientation of the subject.

The second unit 218 may in some embodiments be thought of as an energy delivery unit (EDU), in which case it would incorporate a defibrillator 173, pacer 172, or other electrical therapy 174. In some embodiments, the EDU would be small and light enough to be worn in a harness or belt to be carried around continuously by the subject. The EDU 218 may in some cases not contain a therapy processor 171, but be a “dumb” device that requires the controls provided by connection to the processor in the first (CPR) unit, for example, on the defibrillator pad 10, to deliver electrical therapy to the subject.

Many other embodiments of the invention other than those described above are within the invention, which is defined by the following claims. For example, the defibrillation pads 10, 12 may be separable from the CPR-prompting first unit and be connected at the time that the EDU is brought to the scene; the defibrillation pads may be connected both electrically and mechanically to the CPR-prompting first unit at that time. A greater amount of the control functionality may be put into the first unit, leaving essentially only the circuitry for providing the defibrillation pulses in the second unit. The first unit may be incorporated into the defibrillation electrode pad assembly, or made a separate unit connected to the pad assembly by one or more cables. The second unit may connect to the first unit by one or more cables, or by a wireless connection. The defibrillation pulses may pass through the first unit, or be routed directly to the defibrillation electrodes via one or more cables running from the second unit to the electrodes. The second unit may connect to the first unit by being plugged into the first unit, without the need for a cable (for example, the second unit could be a defibrillation module that plugs into the first unit).

In some embodiments the second (therapy) unit can provide pacing therapy as well as defibrillation therapy. Pulse detection methods other than pulse oximetry, impedance sensing, impedance tomography, or a phonocardiogram, for example, a blood pressure monitor 188 may be employed. Any method capable of detecting a subject's pulse can be used for pulse detection.

Having thus described several aspects of at least one embodiment of this invention, it is to be appreciated various alterations, modifications, and improvements will readily occur to those skilled in the art. For example, it is to be appreciated that any of the features of any of the embodiments disclosed herein may be combined or substituted for features of any other embodiment disclosed herein. Such alterations, modifications, and improvements are intended to be part of this disclosure, and are intended to be within the scope of the invention. Accordingly, the foregoing description and drawings are by way of example only.

What is claimed is:

1. A resuscitation device comprising:
a displacement monitor;
a blood perfusion monitor; and
a processor in communication with the displacement monitor and the blood perfusion monitor, the processor configured to initiate a measurement of blood perfusion using the blood perfusion monitor responsive to a signal received from the displacement monitor.

2. The resuscitation device of claim 1, wherein the resuscitation device is configured to be disposed on a chest of a subject, and wherein the processor is further configured to:
analyze the signal received from the displacement monitor;
determine a point in a CPR compression-relaxation cycle being performed on the subject based upon a result of the analysis; and
initiate the measurement of blood perfusion responsive to a determination that the point in the CPR compression-relaxation cycle is at a predefined point.

3. The resuscitation device of claim 2, wherein the predetermined point corresponds to the chest of the subject being in a relaxed state.

4. The resuscitation device of claim 3, wherein the processor is further configured to initiate a second measurement of blood perfusion responsive to a determination that the CPR compression-relaxation cycle is at a second point, the second point corresponding to the chest of the subject being in a compressed state.

5. The resuscitation device of claim 4, wherein the processor is further configured to prompt a CPR administrator to check a pulse of the subject responsive to a result of a comparison between the first measurement of blood perfusion and the second measurement of blood perfusion.

6. The resuscitation device of claim 3, wherein the processor is further configured to prompt a CPR administrator to check a pulse of the subject responsive to a result of the first measurement of blood perfusion.

7. The resuscitation device of claim 1, wherein the displacement monitor comprises an accelerometer.

8. The resuscitation device of claim 1, wherein the blood perfusion monitor is disposed on a portion of the resuscitation device to which chest compressions are applied during administration of CPR.

9. The resuscitation device of claim 8, wherein the blood perfusion monitor comprises an optical pulse oximetry system.

10. The resuscitation device of claim 9, wherein the optical pulse oximetry system comprises a plurality of optical sensors disposed at different locations on the portion of the resuscitation device to which chest compressions are applied during administration of CPR.

11. The resuscitation device of claim 8, wherein the blood perfusion monitor comprises a plurality of electrodes configured to electrically contact skin of the subject and the device includes circuitry configured to determine an impedance between at least one pair of the plurality of electrodes.

12. The resuscitation device of claim 1, further comprising an electrode pad including a pair of defibrillation electrodes, the displacement monitor, and the blood perfusion monitor.

13. The resuscitation device of claim 1, wherein the displacement monitor, the blood perfusion monitor, and the processor are integrated in an integral CPR assistance and feedback device.

14. A method of treating a subject using a resuscitation device, the method comprising:
determining at least one point in a CPR compression-relaxation cycle at which to measure perfusion of the subject, the CPR compression-relaxation cycle including a compression phase and a relaxation phase;
monitoring information indicative of displacement of a chest of the subject during performance of the CPR compression-relaxation cycle using the resuscitation device; and
measuring the perfusion of the subject using the resuscitation device in response to the monitored information corresponding to the at least one point in the CPR compression-relaxation cycle.

15. The method of claim 14, wherein the at least one point corresponds to the chest of the subject being in a relaxed state.

16. The method of claim 15, further comprising:
instructing a CPR administrator to temporarily delay application of another CPR compression-relaxation cycle prior to measuring the perfusion.

17. The method of claim 14, wherein determining the at least one point includes determining a first point corresponding to the chest of the subject being in a relaxed state and determining a second point corresponding to the chest of the subject being in a compressed state.

18. The method of claim 14, further comprising prompting a rescuer to check a pulse of the subject responsive to a result of the measurement of perfusion.

19. The method of claim 14, wherein measuring the perfusion of the subject comprises monitoring the perfusion of the subject at a plurality of points in the CPR compression-relaxation cycle.

20. The method of claim 19, wherein measuring the perfusion of the subject further comprises determining a difference in perfusion between at least two of the plurality of points in the CPR compression-relaxation cycle.

21. The method of claim 14, wherein measuring the perfusion of the subject comprises performing a measurement of an impedance across a portion of skin of the chest of the subject.

22. The method of claim 14, further comprising delivering a defibrillation shock to the subject.

23. The method of claim 14, wherein measuring the perfusion of the subject comprises monitoring the perfusion of the subject at a plurality of physical locations on the subject.

24. The method of claim 23, wherein a first physical location of the plurality of physical locations is proximate a center of a CPR compression region and a second physical location of the plurality of physical locations is proximate a periphery of the CPR compression region.
